(19) United States
(12) Patent Application Publication
(10) Pub. No.: US 2004/0236282 A1
(43) Pub. Date: Nov. 25, 2004

(54) Unit dosage powder container

(76) Inventor: Philip Braithwaite, Tewkesbury (GB)

Correspondence Address: Charlton Shen, 125 Summer Street, Boston, MA, 02110-1618, US

(21) Appl. No.: 10/479,940

(22) PCT Filed: Jun. 5, 2002
(86) PCT No.: PCT/GB02/02301

(30) Foreign Application Priority Data
Jun. 7, 2001 (GB) 0113881.7

(51) Int. Cl. A61M 5/178

(52) U.S. Cl. 604/158

(57) ABSTRACT

There is described a unit dosage powder receptacle which receptacle is provided with a frangible sealing member characterised in that the receptacle is provided with means to enable the frangible sealing member to be ruptured by inserting a powder flow channel through the sealing member such that the sealing member ruptures in a direction away from the powder. There is also described a rupturing member adapted for rupturing such a unit dosage powder receptacle and also a powder delivery device comparing a unit dosage powder receptacle and a method of treatment related thereto.

This invention relates to a novel form of drug container and to medical devices and methods of treatment utilising such containers.

Conventionally known powder inhalation devices comprise a medicament housed in a foil covered blister. In use, the foil and the blister are both ruptured, allowing the powdered medicament to be blown or sucked out However, such systems suffer from the disadvantage that, inter alia, powdered medicament can be entrapped in or around the ruptured foil or in the blister space. This can lead to a patient receiving an inconsistent dosage of medicament.

U.S. Pat. No. 4,718,054 describes a blister pack, e.g. for housing a powdered medicament, which overcomes or mitigates the disadvantages experienced with prior art blister packs by regulating the ratio of the diameter to depth of each of the blisters.

More recent developments have attempted to overcome this problem by providing, for example, a preformed cup which is sealed with a foil strip. In such a system the foil strip is peeled away, rather than pierced or punctured, so as to enable access to the powdered medicament. Such a system is available in the Accuhaler™ device, available in the UK from Glaxo Smith Kline. However, the Accuhaler still suffers from the disadvantage that it is generally inefficient insofar as, inter alia, inspiratory flow may not be directed effectively onto the metered powder mass to ensure that the powder receptacle is emptied adequately and powder may be displaced from the metering receptacle prior to inhalation.

U.S. Pat. No. 5,921,237 to Dura, describes an inhaler comprising a rotatably mounted blister pack disk with means adapted to shear open a sealed blister and thereby deliver the drug dose to the patient.

Similarly International Patent application No. WO 01/72605 to Dura describes a dose strip for use with a powder inhaler which includes a base strip having spaced apart blisters and a lid strip which is attached over the base strip. Lid tabs are attached to the lid strip over each blister and a peel strip is pulled away from the base strip and lid strip, causing a lid tab to shear open the lid strip.

Whilst the Dura prior art documents go some way to overcoming the problems with e.g. the powdered drug being trapped by the ruptured foil, the Dura inhaler and/or blisters suffer from the disadvantage that the delivered does of medicament is inefficient and the device exhibits a high powder retention.

Thus, there has long been a need for an improved inhalation system which, in particular, is efficient in emptying a powder receptacle and maximising the effect of the airflow in aerosolising the powder.

Our co-pending International Patent application No. WO 01/30430 describes a medicament powder cartridge system which comprises, inter alia, a medicament holding chamber which forms a slidable fit within a sleeve.

As hereinbefore describe, the main problem with prior art foil covered receptacles is that inter alia the foil punctures towards the powder, increasing the risk of the powder being trapped under or around the ruptured foil.

We have developed a novel powder delivery system which comprises a foil covered medicament powder receptacle which is adapted to be ruptured by inserting at least one powder flow channel in intimate contact with or adjacent to the powder and when the foil is ruptured, it ruptures “away” from the powder. It is a particular aspect of the present invention that this may be achieved even when a member is used to rupture the foil from the outside.

U.S. Pat. No. 5,562,918 describes a drug capsule comprising a sleeve with open ends, each of which is provide with an end cap. The cap(s) are able to be ruptured by the sleeve thus enabling the drug to be delivered. However, one disadvantage of such a system is that a “flap” of the reservoir remains which, as with many other prior art systems, e.g. conventional blister strips, can entrap and/or hinder powder flow.

DE 19757207 describes a blister pack assembly for an inhaler wherein the blister pack element comprises a plurality of blisters, each containing a dose of medicament powder. In use a suction tube pierces the blister by rupturing the foil. Such a device also suffers from the disadvantage that a flap of the blister remains and which can entrap or hinder powder flow.

Thus, according to the invention we provide a unit dosage powder receptacle, which is provided with a frangible sealing member characterised in that the receptacle is provided with means to enable the frangible sealing member to be ruptured by inserting an powder flow channel through the sealing member such that the sealing member ruptures in a direction away from the powder.

A particular aspect of the present invention enables the powder to be held in a plane which is substantially perpendicular to the plane of the frangible sealing member.

In one embodiment of the invention, the unit dosage powder receptacle as hereinbefore described comprises a unit dosage of, e.g. medicament powder held in a powder reservoir provided with a powder protector. The powder protector is distinct from the frangible sealing member, preferably the powder protector is slidable to enable it to be moved from a protecting position to an unprotecting position. In a preferred embodiment the powder reservoir and the powder protector both lie in a plane perpendicular to the plane of the frangible sealing member. In an especially preferred embodiment the powder reservoir and powder protector comprises a spool/spool sleeve assembly, such as is described in European Patent No 0 626 869 which is incorporated herein by reference.

Thus, the spool/spool sleeve may be housed in a spool receptacle, the receptacle may, for example, be frusto conical in shape. Thus the spool/spool sleeve may be positioned such that one end is positioned adjacent the frangible sealing member, e.g. foil and the second end is positioned adjacent the base of the receptacle.

The frusto conical nature of the receptacle is such that the second end of the spool may form a snug fit whilst the conical sides slope away from the first end of the spool.

Furthermore, the second end of the spool is located against a spigot, said spigot may be separate or may be integral to either the spool or the receptacle. The receptacle may be provided with a recessed portion in which the spigot may sit The recessed portion is preferentially such that it will form a snug fit with the spool sleeve. Further, the length/depth of the recess should be not less than similar to the length of the spool sleeve. In an analogous manner the length of the spigot should be similar to the length of the spool sleeve. Thus, when the spool sleeve is urged away from the spool, the spool will remain static and the sleeve may slide into the recess exposing the spool and/or powder. Preferably, the sleeve is urged away so as to expose the whole of the powder and/or spool. In a particular aspect of the invention, at the point when the powder and/or spool are exposed, at least one powder channel is placed in intimate contact with the powder and/or spool.

The dimensions of the powder chamber may be varied, permitting different dosages of powder to be administered. Preferably, the dimensions are such that the chamber will be filled to provide a single desired dose. In one embodiment the chamber is a substantially elongate member e.g. a cylindrical member with an open end and a closed end. When the closure member comprises a removable cap, the cap may rest on the sleeve. However, preferentially, the chamber may be provided with one or more spacers at its open end. Preferably at least two spacers are present to allow even resting of the cap. The use of spacers is advantageous in that they prevent the cap from coming into contact with the powder and possibly reducing the accuracy of the dosage delivered. The spacers can also act to enhance removal of the cap. The spacers may optionally be provided with a ridge upon which the cap may rest.

The cap may generally be the same diameter as the powder chamber. The cap may comprise a flat disc, a plug or an inverted cup. It is desirable that the cap should provide a closed face abutting the chamber and/or spacers. The length of the cap will be small relative to the length of the chamber.

The sleeve is adapted to form a snug fit at least around the joint formed between the open end of the container and the cap. The sleeve preferentially wraps around the whole of the circumference of the joint so as to form a seal. The sleeve comprises a substantially resilient material, e.g. a plastics sleeve, in order for the inner walls of the sleeve to be biased towards the joint so as to form a sealing engagement. In a preferred embodiment a longitudinal sleeve is used enabling it to also act as a support for the body of the chamber. Thus, it is preferred that the length of the sleeve will be substantially the same as the length of the chamber. In an especially preferred embodiment the length of the sleeve is such that it forms a snug fit with the chamber with only the spacers protruding from the sleeve. When the cap is placed upon the spacers and urged against the chamber so that the container is partially pushed through the sleeve, the closed end of the container protruding from the sleeve and the sleeve forming a sealing engagement with the cap and the container.

When the closure member comprises a sealing member, the sealing member is preferably fixed to the sleeve. The sealing member will generally be the same diameter as the sleeve or, optionally, it may be of slightly greater dimensions such that it overlaps the end of the sleeve. The sealing member may preferentially comprises any frangible material. Materials which are impermeable to moisture and/or are moisture resistant are preferred. Such materials include, but are not limited to, plastics films or foils, e.g. aluminium foil material. In the case of a plastics sealing member, this may simply be heat bonded to the sleeve, whilst with a foil sealing member, a layer of conventionally used adhesive may be used to bond the foil to the sleeve.

The delivery device of the invention may utilise any conventionally known means of puncturing or rupturing the frangible sealing member, e.g. foil strip. Preferentially, the rupturing tool is adopted to perform the dual function of rupturing the frangible member and separately, simultaneously or sequentially urging the separation of the spool and the spool sleeve. Preferably, this comprises urging the spool sleeve away from the spool.

It is a further aspect of the invention to provide a rupturing or cutting member which is novel per se.

In this aspect of the invention we especially provide a rupturing member adapted for rupturing the frangible sealing member of a unit dosage powder receptacle as hereinbefore described characterised in that the rupturing member is adapted to act as a rupturing member, a spool/spool holder separator and a powder channel.

In a preferred embodiment the rupturing member may achieve this by being provided with one or more surfaces which are adapted to be coincident with the walls of the receptacle, especially the inclined region of the receptacle walls. Preferentially, the inclined region of the rupturing member may form an edge of the cutting point.

In use the rupturing member may be removed to expose the spool and/or powder. Alternatively, the rupturing member may be provided with one or more conduits which is adapted to form a passageway from the spool to a powder delivery channel for the powder. Thus, the rupturing member may also comprise one or more powder channels, preferably a pair of powder channels. In an especially preferred embodiment the conduit may be adapted to overlie all or part of the powder when the rupturing member has ruptured the frangible sealing member.

The dosage unit may also be one of a plurality of such units arranged in series, which units are able to transfer a succession of metered doses of powder into the inhalation passage of a dry powder inhaler. When a plurality of dosage units are connected together, the sleeves required may be comprised of a cartridge with a plurality of sleeves arranged around its periphery. In such a case the dosage units themselves may be connected together or it may be that the sleeves are connected together, or both.

The invention thus also provides a plurality of dosage units arranged in series, each unit being as hereinbefore described. The units may be releasably or permanently attached to one another so as to be in a chain-like conformation, preferably a flexible or semi-flexible chain. The design of dosage units in accordance with the invention makes such flexibility possible.

A series of dosage units in accordance with this aspect of the invention is ideal for use in an inhaler, because it allows sequential presentation of doses of a powdered medicament to the inhalation passage of the inhaler as the series is indexed through the inhaler. If the series is in the form of a flexible chain, it can then be rolled or folded up for compact storage in the inhaler. The series may be of any appropriate length. It may, for instance, be supplied in a length greater than might be needed for use in an inhaler, but capable of being broken up into usable lengths. In an especially preferred embodiment the plurality of dosage units are contained in a cartridge and such a cartridge forms a further aspect of the invention.

In use, when placed in an inhaler, such as the TECHNOHALER, a push rod can act upon the closed end of the container protruding from the sleeve, urging the container back in the sleeve, and causing the cap to be ejected from the other end of the sleeve. When the container is in the inverted position, that is, the closed end uppermost, the cap falls away and the container empties the powdered medicament into the inhalation passage of the inhaler.

Thus, according to a further feature of the invention we provide a powder delivery device comprising a powder dosage unit as hereinbefore described. In a most preferred embodiment the powder is a medicament and therefore, preferably, the delivery device is a medicament delivery device, such as an inhaler, e.g. a dry powder inhaler.

Thus, according to a further feature of the invention we provide a dry powder inhaler comprising medicament and a dosage unit as hereinbefore described. In a further embodiment we provide an inhaler as hereinbefore described comprised a plurality of medicament dosage units.

When the powder delivery device comprises an inhaler the powder channel of the rupturing member may comprise an air channel and/or an aerosolisation channel. Whilst, generally, the powder/air channel in the rupturing member is adapted for the removal of powder, e.g. in aerosolised form, from the metering member, it may also be used to introduce, e.g. flushing air in the powder receptacle.

In the inhaler of the invention the medicament dosage units are preferably presented in a cartridge as hereinbefore described.

A variety of medicaments may be administered by using the inhaler of the invention. Such medicaments are generally suitable for the treatment of asthma, COPD and respiratory infections. Such medicaments include, but are not limited to β₂-agonists, e.g. fenoterol, formoterol, pirbuterol, reproterol, rimiterol, salbutamol, salmeterol and terbutaline; non-selective beta-stimulants such as isoprenaline; xanthine bronchodilators, e.g. theophylline, aminophylline and choline theophyllinate; anticholinergics, e.g. ipratropium bromide; mast cell stabilisers, e.g. sodium cromoglycate and ketotifen; bronchial anti-inflammatory agents, e.g. nedocromil sodium; and steroids, e.g. beclomethasone dipropionate, fluticasone, budesonide, flunisolide and ciclesonide, and isomers and/or salts or derivatives thereof.

Specific combinations of medicaments which may be mentioned include combinations of steroids, such as, beclomethasone dipropionate and formoterol; beclomethasone dipropionate and salmeterol; fluticasone and formoterol; fluticasone and salmeterol; budesonide and formoterol; budesonide and salmeterol; flunisolide and formoterol; and flunisolide and salmeterol. It is also within the scope of this invention to include combinations of one or more of the aforementioned steroids with one or more of the aforementioned β₂-agonists.

Further medicaments which may be mentioned include systemically active materials, such as, proteinaceous compounds and/or macromolecules, for example, hormones and mediators, such as insulin, human growth hormone, leuprolide and alpha interferon, growth factors, anticoagulants, immunomodulators, cytokines and nucleic acids.

According to a further aspect of the invention we provide a method of delivering a powder which comprises the use of a powder delivery device as hereinbefore described.

We further provide a method of treatment of a patient with a respiratory disorder which comprises the administration of a medicament using a powder delivery device as hereinbefore described.

We also provide a method of treatment of a patient with a systemic disorder which comprises the administration of a medicament using a powder delivery device of the invention.

The invention will now be described by way of example only and with reference to the accompanying drawings in which,

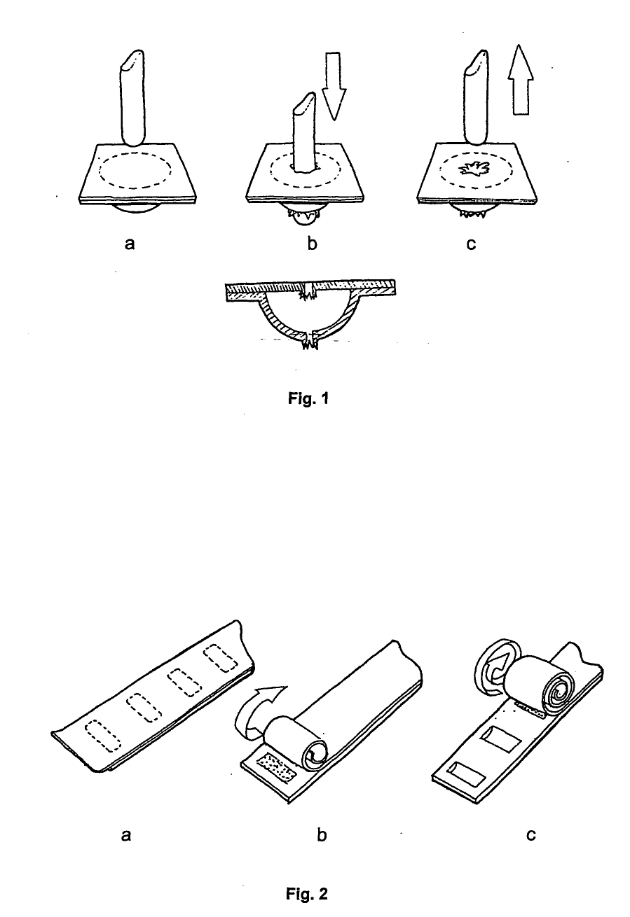

FIGS. 1a to d are representations of a prior art system of rupturing a blister using a rupturing tool;

FIGS. 2a to c are representations of a prior art system of peeling a foil cover from a blister;

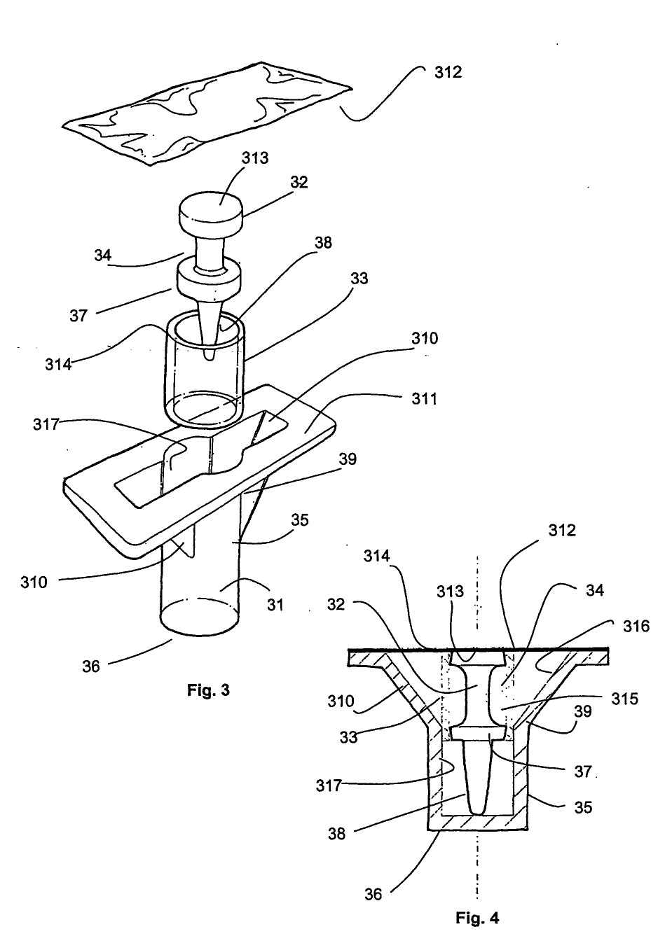

FIG. 3 is a perspective view of a disassembled dosage unit wherein a spool is provided with an integral spigot;

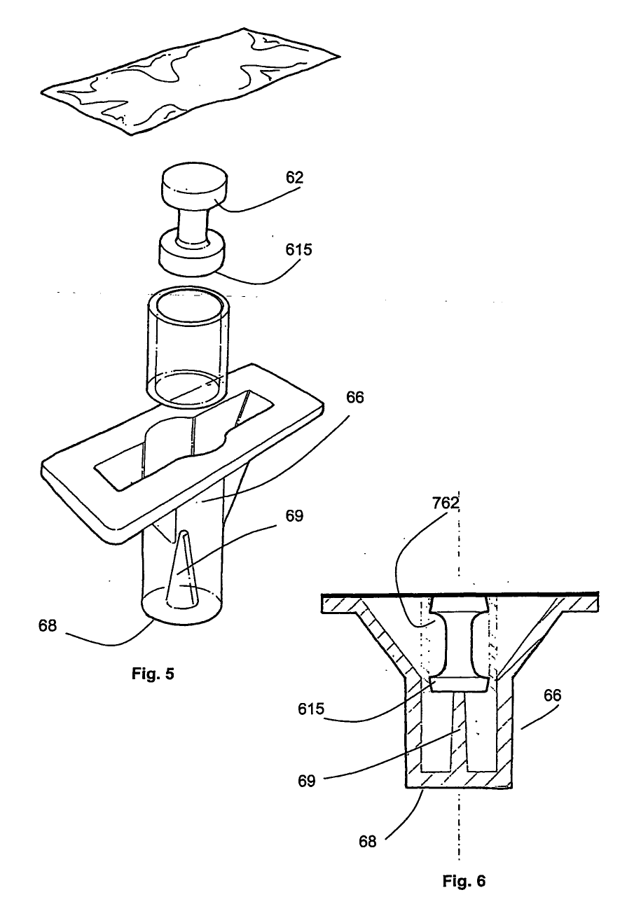

FIG. 4 is a cross-sectional view of an assembled dosage unit wherein a spool is provided with an integral spigot;

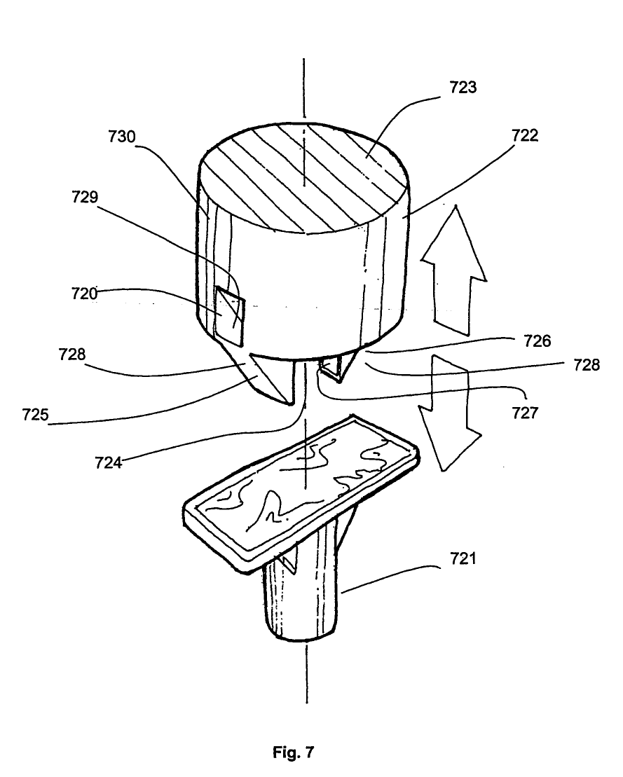

FIG. 5 is a is a perspective view of a disassembled dosage unit wherein a receptacle is provided with an integral spigot;

FIG. 6 is a cross-sectional view of an assembled dosage unit wherein a receptacle is provided with an integral spigot;

FIG. 7 is a perspective view of a dosage unit and a foil cutting or rupturing tool;

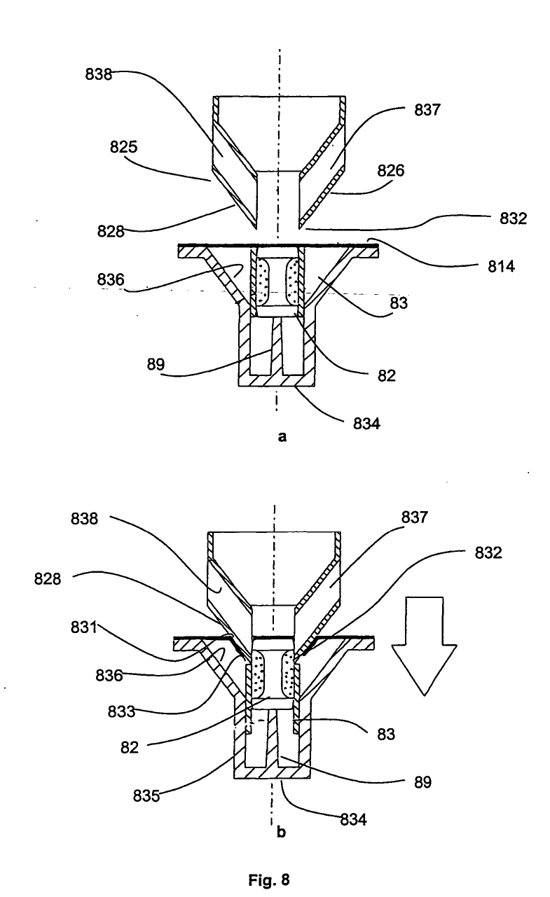

FIGS. 8a and b are cross-sectional views of a dosage unit and a foil cutting or rupturing tool in use rupturing a foil cover,

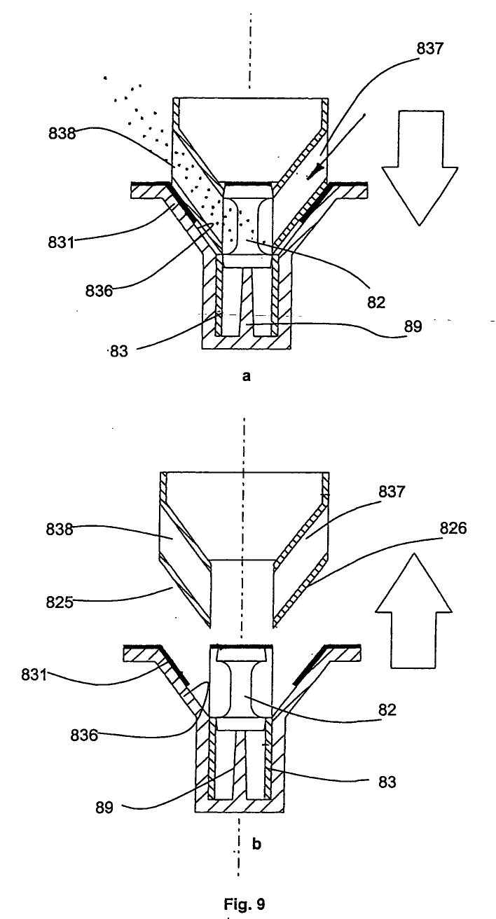

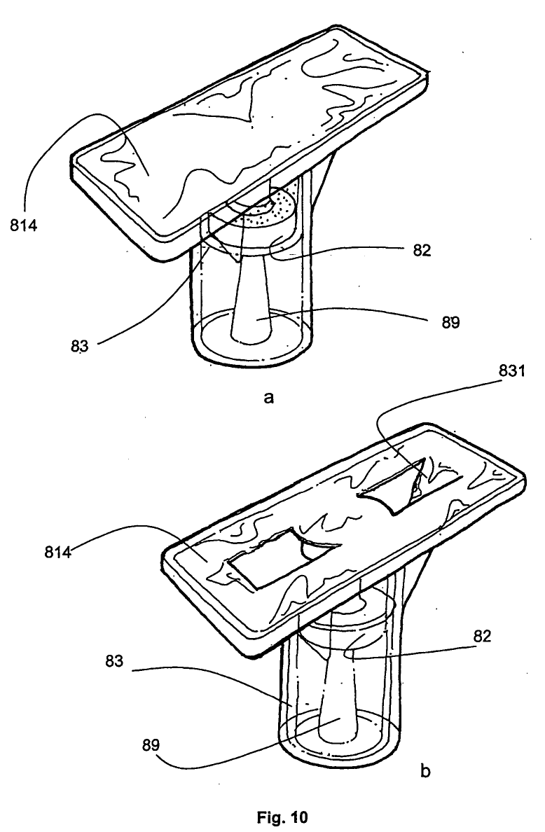

FIGS. 9a and b are cross-sectional views of a dosage unit and a foil cutting or rupturing tool in use in position to deliver medicament powder and being removed after use;

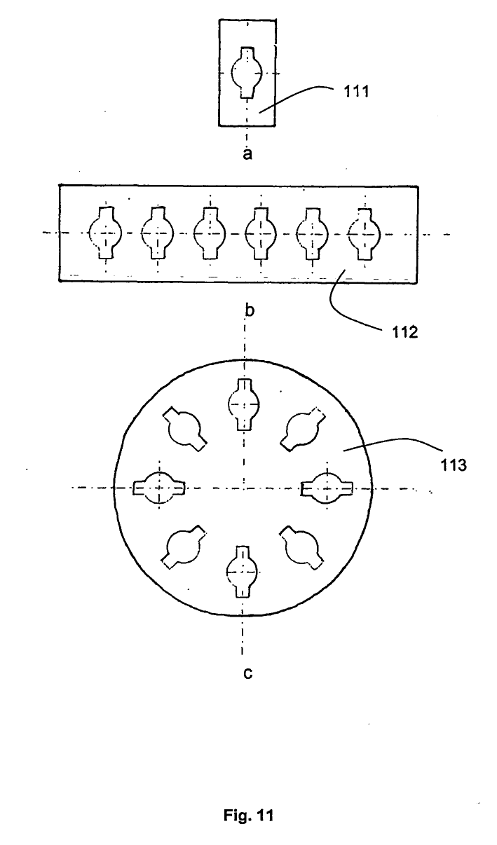

FIGS. 10a and b are perspective views of a sealed dosage unit before and after use; and

FIGS. 11a to c are schematic representations of a single dosage unit and a plurality of dosage units in strip form and cartridge form.

FIGS 1a-1d illustrate a blister and piercing tool of the prior art. The foil, when ruptured, can form folded pockets which can entrap powder which is unable to escape.

FIGS. 2a-2c illustrate a peelable foil blister strip of the prior art. The system avoids piercing of the foil strip, but the efficiency of emptying the blister may be limited.

Referring to FIGS. 3 and 4, a unit dosage powder receptacle (31) comprises a spool (32) and a sleeve (33) in the form of a spool holder. The spool (32) and spool holder (33) together form a medicament powder reservoir (34). The receptacle (31) comprises a cylinder (35) adapted to form a snug fit with the sleeve (33). The cylinder (35) has a base (36) and one end (37) the spool (32) is provided with a spigot (38) which rests on the base (36). The end (39) of the receptacle (31) distal to the base (36) comprises of a recessed portion (310). The recessed portion (310) being angled inwards towards the base (36) of the receptacle (31) so that an upward facing inclined surface (316) is provided at either side of the receptacle (31). The end (39) of the receptacle (31) is provided with a sealing end face (311). The receptacle is provided with a foil sealing strip (312).

In use, one end (37) of the spool (32) rests on the spigot (38) and a second end (313) of the spool (32) abuts the inner surface of the foil strip (312). The end (314) of the sleeve (33) also abuts the inner surface of the foil strip and the sleeve (33) forms a snug fit with the inner wall (317) of the receptacle (31).

Medicament powder (315) is housed between the spool (32) and the spool holder (33).

Referring to FIGS. 5 and 6, the assembly is analogous to that hereinbefore described in FIGS. 3 and 4. However, the base (68) of the receptacle (66) is provided with a spigot (69). The end (615) of the spool (62) is substantially flat and rests on the spigot (69) at the base of the receptacle (66).

Referring to FIG. 7, the system is provided with a sealed single dosage unit (721) and a foil cutting or rupturing member (722). The foil cutting member (722) comprises a cylinder (723), the dosage unit facing surface (724) of the cylinder (723) is provided with a pair of teeth (725 and 726). The teeth (725 and 726) are spaced apart precisely the same dimensions as the diameter of the spool holder (not shown in FIG. 7). The inner, facing surfaces, (727) of the teeth (725 and 726) are hollow so that an “open” surface is present The distal, outer surfaces (728) are inclined so as to correspond with the inclined surface of the receptacle.

Each of the hollow teeth (725 and 726) separately form a conduit (729) from the open surface, (727) to an aperture (720) on the outer wall (730) of the cylinder (723). The conduit (729) comprises a powder passage.

Referring to FIGS. 8 to 10, in use the teeth (825 and 826) pierce the foil strip (814). The ruptured portion (831) of the foil (814) is biased away from the powder (818), by the inclined surfaces (828) of the teeth (825 and 826).

The cutting point (832) of each of the teeth (825 and 826) engages with the upper end surface (833) of the spool holder/sleeve (83). At the same time, the further ingress of the cutting member (822) slides the spool carrier (83) away from the spool (82) towards the base (834) of the receptacle (835). The spool (82) which is supported by the spigot (89) remains stationary.

As the cutting member further ingresses, into the receptacle the sleeve (83) is pushed to the base (834), exposing the spool (82) and the powdered medicament to the open face of the teeth (825 and 826). The inclined wall (828) of teeth (825 and 826) mates with the inclined wall (836) of the receptacle and also pushes the ruptured portion (831) of the foil against the inclined wall (836) thus removing the possibility of any contact between the foil (831) and the medicament.

Air may then enter (or alternatively a vacuum may be used) through one of the conduits (837) of the teeth (825 and 826) to deagglomerate and aerosolise the medicament, which will exit through the other conduit (838).

The cutter is then retracted from the receptacle and is primed for the introduction of a new filled spool.

Referring to FIGS. 11 a to c, a single dosage unit (111) may be linked together to provide a plurality of dosage units in strip form (112) or cartridge form (113).

1. A unit dosage powder receptacle (31) with receptacle is provided with a frangible sealing member (312) receptacle is provided with means to enable the frangible sealing member (312) to be ruptured by inserting a powder flow channel (729) through the sealing member (312) characterised in that the sealing member ruptures in a direction away from the powder.

2. A unit dosage powder receptacle (31) according to claim 1 characterised in that the powder is held in a powder reservoir (34) in a plane which is substanially perpendicular to the plane of the frangible sealing member (312).

3. A unit dosage powder receptacle (31) according to claim 1 characterised in that the powder is held in a powder reservoir (34) provided with a powder protector (33).

4. A unit dosage powder receptacle (31) according to claim 3 characterised in that the powder reservoir (34) and powder protector (33) comprises a spool/spool sleeve assembly (34 and 33).

5. A unit dosage powder receptacle (31) according to claim 4 characterised in that the spool/spool sleeve assembly (34 and 33) comprises an assembly as described in European Patent No. 0 626 869.

6. A unit dosage powder receptacle (31) according to claim 4 characterised in that the spool/spool sleeve assembly (33 and 34) is housed in a spool receptacle.

7. A unit dosage powder receptacle according to claim 5 characterised in that the receptacle is substantially frusto conical in shape.

8. A unit dosage powder receptacle according to claim 4 characterised in that the spool/spool sleeve is positioned such that a fit end is positioned adjacent the frangible sealing member and a second end is positioned adjacent the base of the recycle.

9. A unit dosage powder receptacle according to claim 8 charaterised in that the frusto conical nature of the rectangle is such that the second end of the spool forms a snug fit with the receptacle while the conical sides of the receptacle slope away from the first end of the spool.

10. A unit dosage powder receptacle according to claim 9 characterised in that the second end of the spool is located against a spigot.

11. A unit dosage powder receptacle according to claim 10 characterised in that the spigot is integral to the spool.

12. A unit dosage powder receptacle according to claim 10 charaterised in that the spigot is integral to the receptacle.

13. A unit dosage powder receptacle according to claim 10 charaterised in that the receptacle is provided with a recessed portion in which the spigot sits.

14. A unit dosage powder receptacle according to claim 14 charaterised in that the recessed portion also forms a snug fit with the spool sleeve.

15. A unit dosage powder receptacle according to claim 14 charaterised in that the depth of the recess is substantially similar to the length of the spool sleeve.

16. A unit dosage powder receptacle according to claim 1 characterised in that the dimensions are such that the powder reservoir is filled to provide a single desired dose.

17. A unit dosage powder receptacle according to claim 4 characterised in that the spool and/or the spool sleeve substantially abuts the frangible sealing member.

18. A unit dosage powder receptacle according to claim 17 characterised in that the frangible sealing member is fixed to the chamber.

19. A unit dosage powder receptacle according to claim 1 characterised in that the frangible sealing member comprises a material which is substantially impermeable to moisture and/or is moisture resistant.

20. A unit dosage powder receptacle according to claim 19 characterised in that the frangible sealing member comprises a plastics film or foil.

21. A unit dosage powder receptacle according to claim 20 characterised in that the frangible sealing member comprises an aluminium foil material.

22. A unit dosage powder receptacle according to claim 20 characterised in that the frangible sealing member comprises a plastics material that is heat bonded to the receptacle.

23. A unit dosage powder receptacle according to claim 21 characterised in that the frangible seating member comprises a foil material that is bonded to the receptacle with an adhesive.

24. A rupturing member adapted for rupturing the frangible sealing member of a unit dosage powder receptacle according to claim 1 characterised in that the rupturing member is adapted to act as a rupturing member, a spool/spool holder separator and a powder channel.

25. A rupturing member according to claim 24 characterised in that the rupturing member is adapted to rupture the frangible sealing member in a direction away from the powder.

26. A rupturing member according to claim 24 characterised in that the rupturing member is provided with one or more surfaces which are adapted to be coincident with the walls of the receptacle.

27. A rupturing member according to claim 26 characterised in that the rupturing member comprises a pair of inclined walls adapted to be coincident with the walls of the inclined region of the receptacle.

28. A rupturing member according to claim 27 characterised in that the end of the inclined walls of the rupturing member may form a cutting point.

29. A rupturing member according to claim 24 characterised in that the rupturing member is provided with one or more conduits which act as a powder conduit.

30. A rupturing member according to claim 29 characterised in that the rupturing member is provided with a pair of powder conduits.

31. A rupturing member according to claim 29 characterised in that the rupturing member is adapted for the introduction of flushing air into the powder receptacle.

32. A powder delivery device which comprises a unit dosage powder receptacle according to claim 1.

33. A powder delivery device according to claim 32 characterised in that the delivery device includes means for rupturing the frangible sealing member.

34. A powder delivery device according to claim 33 characterised in that the rupturing means is also adapted to urge the separation of the spool and the spool sleeve.

35. A powder delivery device according to claim 34 characterised in that the rupturing means is adapted to act on the spool to slidably urge the separation of the spool sleeve away from the spool.

36. A powder delivery device according to claim 33 characterised in that the rupturing means is also adapted to act as a powder channel.

37. A powder delivery device according to claim 36 characterised in that at the point when the powder and/or spool are exposed the powder channel is placed adjacent to or in intimate contact with the powder and/or spool.

38. A powder delivery device according to claim 36 characterised in that the powder conduit of the rupturing member is adapted to overlie all or part of the powder when the rupturing member is fully inserted.

39. A unit dosage powder receptacle according to claim 1 characterised in that a plurality of dosage units are arranged in series.

40. A cartridge comprising a series of unit dosage powder receptacles according to claim 39.

41. A powder delivery device according to claim 29 characterised in that the powder is a medicament.

42. A powder delivery device according to claim 41 characterised in that the delivery device is an inhaler.

43. A powder delivery device according to claim 42 characterised in that the delivery device is a dry powder inhaler.

44. A powder delivery device according to claim 1 characterised in that the as device comprises a plurality of unit dosage powder receptacles.

45. A powder delivery device according to claim 43 characterised in that the powder channel is air channel.

46. A powder delivery device according to claim 41 characterised in that the device is provided with suitable indexing means.

47. A powder delivery device according to claim 41 characterised in that the powder is selected from the group of drugs for the treatment of asthma, COPD or, respiratory infections such as β₂-agonists, e.g. fenoterol, formoterol, pirbuterol, reproterol, rimiterol, salbutamol, salmeterol and terbutaline; non-selective beta-stimulants such as isoprenaline; xanthine bronchodilators, e.g. theophylline, aminophylline and choline theophullinate; anticholinergics, e.g. ipratropium bromide; mast cell stabilisers, e.g. sodium cromoglycate and ketoifen; bronchial anti-inflammatory agents, e.g. nedocromil sodium; and steriods, e.g. beclomethasone dipropionate, fluticasone, budesonide, fluisolide and ciclesonide, and isomers and/or salts or derivatives thereof.

48. A powder delivery device according to claim 47 characterised in that device comprises a combination of medicaments, selected from steroids, such as beclomethasone dipropionate and formoterol; beclomethasone dipropionate and salmeterol; fluticasone and formoterol; fluticasone and salmeterol; budesonide and formoterol; budesonide and salmeterol; flunisolide and formoterol; and flunisolide and salmeterol.

49. A powder delivery device according to claim 41 characterised in that device comprises a systemically active materials, such as, proteinaceous compounds and/or macromolecules, for example, hormones and mediators, such as insulin, human growth hormone, leuprolide and alpha interferon; growth factors, anticoagulants, immumonomodulators, cytokines and nucleic acids.

50. A method of delivering a powder which comprises the use of a powder delivery device according to claim 41.

51. A method of treatment of a patient with a respiratory disorder which comprises the administration of a medicament using a powder delivery device according to claim 42.

52. A method of treatment of a patient with a systemic disorder which comprises the administration of a medicament using a powder delivery device according to claim 41.

53. A unit dosage powder receptacle, rupturing member or powder delivery device substantially as described with reference to the accompanying drawings.
